(19) United States
(12) Patent Application Publication
(10) Pub. No.: US 2006/0235321 A1
(43) Pub. Date: Oct. 19, 2006

(54) ECG filtering

(76) Inventors: Steven John Simske, Fort Collins, CO (US); Daniel Robert Blakley, Philomath, OR (US); Tong Zhang, San Jose, CA (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/107,264

(22) Filed: Apr. 15, 2005

(51) Int. Cl. A61B 5/04

(52) U.S. Cl. 600/512

(57) ABSTRACT

A method of obtaining a filtered electrocardiogram is provided. The method includes the steps of obtaining a vectorcardiogram, filtering the vectorcardiogram to form a filtered vectorcardiogram, and transforming the filtered vectorcardiogram into a filtered electrocardiogram. The method may also include the preliminary step of obtaining an electrocardiogram, and then transforming the electrocardiogram into the vectorcardiogram.

### FIELD OF THE INVENTION

The present invention relates generally to electrocardiogram recordings. More particularly, the present invention relates to the filtering of noise artifacts from electrocardiogram recordings.

### BACKGROUND OF THE INVENTION

Electrocardiogram (ECG) recordings are important indicators used in the diagnosis of many cardiac abnormalities and diseases. The ECG is a graphical representation of the electrical voltage in the heart produced during a cyclical heartbeat. One common ECG method utilizes three leads, Lead I, Lead II, and Lead III. Each lead has a negative and a positive electrode that measure electrical potentials between various points on the body. Lead I measures the electrical potential from the right arm to the left arm, Lead II measures the electrical potential from the right arm to the left leg, and Lead II measures the electrical potential from the left arm to the left leg. From this, three additional “augmented” leads, aV_{R}, aV_{L}, and aV_{F}, measure electrical potentials between a point V located centrally in the chest and each of the three limb leads.

ECG leads measure the average electrical activity generated by the summation of the action potentials of the heart at a particular moment in time. For example, during normal atrial systole, the summation of the electrical activity produces an electrical vector that is directed from the sinoatrial (SA) node towards the atrioventricular (AV) node, and spreads from the right atrium to the left atrium. This directionality is a result of the location of the SA node in the right atrium. This electrical activity is represented by the P wave on the ECG.

One common problem that can often make reading ECGs and subsequent diagnoses difficult is the introduction of noise artifacts into the ECG recording. These noise artifacts, which can be from a variety of sources, including breathing and motion artifacts, DC drift, saturation, and power line noise, often obscure much of the detail of the ECG that may be valuable in the diagnosis of cardiac abnormalities. Filtering the ECG is often difficult, due to the abrupt transitions contained in the QRS wave. Because of this, ECGs are difficult to filter without losing valuable information content. As such, it would be beneficial to develop a means of providing filtered ECGs while maintaining a majority of the information content normally contained therein.

### SUMMARY OF THE INVENTION

It has been recognized that it would be advantageous to provide a filtered electrocardiogram that preserves much of the informational content collected from a subject. As such, the present invention provides a method of obtaining a filtered electrocardiogram comprising the steps of obtaining a vectorcardiogram, filtering the vectorcardiogram to form a filtered vectorcardiogram, and transforming the filtered vectorcardiogram into a filtered electrocardiogram. The method may also include the preliminary step of obtaining an electrocardiogram, and then transforming the electrocardiogram into the vectorcardiogram.

Additional features and advantages of the invention will be apparent from the following detailed description which illustrates, by way of example, features of the invention.

### BRIEF DESCRIPTION OF THE DRAWING

FIG. 1 is a graphical representation of a three-lead electrocardiogram configuration in accordance with an embodiment of the present invention.

### DETAILED DESCRIPTION OF THE PREFERRED EMBODIMENT(S)

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

In describing and claiming the present invention, the following terminology will be used.

The singular forms “a,” “an,” and “the” include plural referents unless the context clearly dictates otherwise. Thus, for example, reference to “a variable” includes reference to one or more of such variables.

As used herein, “electrocardiogram” and “ECG” may be used interchangeably, and refer to recordings of electrical activity of the heart muscle.

As used herein, “vectorcardiogram” and “VCG” may be used interchangeably, and refer to a representation of the magnitude and direction of the electrical activity in the heart in the form of vector loops.

As used herein, “a lead” refers to a pair of electrodes utilized to measure the electrical potential between two locations on the body.

As used herein, “signal” and “waveform” may be used interchangeably, and refer to a representation of the flow of information through a lead. It is also intended that these terms include a representation, graphical or otherwise, of a single ECG or VCG, or multiple ECGs or VCGs.

As used herein, “signal artifact” and “noise artifact” may be used interchangeably, and refer to undesirable signal contamination that may or may not obscure ECG or VCG information content.

Physicians and other medical professionals typically diagnose cardiac pathologies using ECGs rather then the related, and often more diagnostically valuable, VCGs. There may be a number of reasons for this preference, including the relatively simpler nature of the ECG curves, greater familiarity with ECGs, and the typical need for a mathematical transformation to obtain the VCG. VCGs, however, have smoother curves than ECGs, making noise artifact removal via filtering much more straightforward. The present invention provides a method for filtering a VCG to remove noise and other artifacts, followed by a transformation of the VCG to an ECG in order to provide physicians and other medical professionals with cardiac data in a more familiar form. As such, filtered VCGs can be used to regenerate any standard lead signal (e.g., 3-lead signal, 12-lead signal, etc.), resulting in a much improved ECG signal.

The present invention provides a method of obtaining a filtered ECG, including the steps of obtaining a VCG, filtering the VCG to form a filtered VCG, and transforming the filtered VCG into a filtered ECG. The method can also include the preliminary steps of obtaining an ECG, and transforming the ECG into the VCG. In one aspect, the ECG can be obtained as a raw ECG. A raw ECG is an ECG that has not been filtered, compressed, or processed, or, in other words, an ECG that is in essentially the same form as originally recorded and/or stored in a storage location. In another aspect, the ECG can be obtained as a processed ECG. A processed ECG is an ECG that has undergone some amount of processing, such as, but not limited to, filtering, compression, transformation, or combinations thereof. It is intended that any means of obtaining a VCG known to one skilled in the art be included within the scope of the present invention, whether it be through transformation of a raw or processed ECG, obtaining a VCG from a storage location, or through the direct recording of a VCG from a patient.

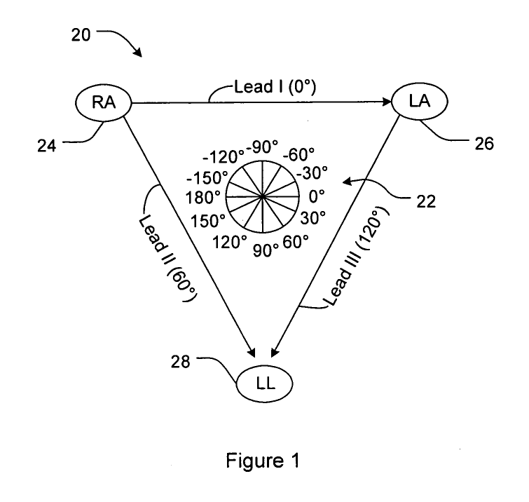

In one aspect of the present invention, the step of obtaining the ECG can further comprise the steps of electrically associating at least two leads with a subject, and recording ECG signals from the subject via the at least two leads. In one aspect, electrically associating a lead with a subject would comprise attaching a positive electrode and a negative electrode to a subject at distinct locations. As is well know in the art, a single electrode can function as an electrode for more than one lead. As shown in FIG. 1, one common method of recording an ECG signal, as described above, utilizes a three-lead relationship 20. Electric potentials between any two electrodes comprising a lead can be recorded as an ECG. So, for the three-lead example comprising Leads I, II, and III, a recording in Lead II is the sum of the recordings in Leads I and II. These three leads provide the basis for a clockwise polar coordinate system 22 in which angle 0° is along Lead I, and thus Lead I is at 0°, Lead II is at 60°, and Lead III is at 120°. In FIG. 1, Lead I measures electrical potentials between the right arm 24 and the left arm 26, Lead II measures electrical potentials between the right arm and the left leg 28, and Lead III measures electrical potentials between the left arm and the left leg. This configuration should not, however, be seen as limiting to the present invention. As such, in one aspect, the step of electrically associating at least two leads with a subject can include electrically associating at least three leads. In another aspect, the step of electrically associating at least two leads with a subject can include electrically associating at least four leads.

Given the differential placement of the leads on the body, specific leads may produce signals of varying quality. Factors determining the differential quality that may exist between leads can include local motion, endogenous biological signals such as muscular noise, line noise, etc. It can be beneficial to select a pair of leads that provides an acceptable level of signal quality for transforming the ECG into the VCG. In many cases it may be preferable to select the pair of leads that has a higher level of signal quality than each of the other combinations of pairs of leads, i.e., the pair with the highest level of signal quality. Because many of the types of noise artifacts that may be present in an ECG can be linearly independent and independently identifiable, lead selection can be based on any number of criteria, one of which may include a weighted combination of the prevalence of each distinct noise artifact.

In one aspect, a simpler method of selection of a pair of leads may include estimating what percent of the signal suffers from one or more of the distinct noise artifact(s). Simply taking the signal variance may not be sufficient, for example, because a signal that is fully saturated only on one side of the range will have zero variance, but also zero signal. In such a situation, breaking up the waveform into reasonably sized time windows, e.g., 1000 msec, and assessing whether noise is present may prove beneficial. For example, each time window containing a particular type of noise artifact can be tagged with a “1.” Those signals with noise present will have a higher variance than a “cleaner” signal. As such, leads with a lower variance can be preferentially selected.

In an even simpler aspect, the leads can be prioritized and the best pair of leads selected based only on breathing/motion signal artifacts using the variance methods as described herein. This is due to an assumption that many common signal artifacts can be discounted due to their nature. For example, it can be assumed that DC drift may be irrelevant, because a medical diagnosis does not depend on the DC value and DC values disappear from the VCG anyway. Also, it can be assumed that saturation does not occur because the gain of the recording instrument is not set high enough. Additionally, many common signal artifacts can be removed, further justifying selecting a pair of leads based primarily on breathing/motion artifacts. For example, power line noise can be removed with a 50 or 60 Hz notch filter, depending on the frequency of the noise. DC drift can be removed by applying a high-pass filter to the signal. Segments of the signal having saturation noise artifacts can be discarded as unreliable data.

In one aspect, ECG data can be immediately processed upon recording. Immediately processed upon recording is intended to include simultaneous recording and processing. In other words, the step of transforming the ECG into the VCG can occur during an overlapping period of time with respect to the step of recording of the ECG signal. The overlapping period can be completely overlapping, or merely overlapping for a short period of time. The actual transformation of the ECG into the VCG may be delayed slightly from the recording step due to the manner in which data is processed in the recording apparatus.

In addition to recording an ECG, another aspect of the present invention includes obtaining the electrocardiogram from a storage location. The storage location may include any type of digital or analogue storage known to one skilled in the art, such as, but not limited to, hard disk storage, removable disk storage, tapes, optical disks, flash memory, RAM or other volatile memory, etc. The ECG can be obtained from a workstation, an ECG recording device, a handheld computer, a laptop, a network, a cellular network, or by any other means known to one skilled in the art.

Various methods of transforming an ECG into a VCG may be contemplated by one skilled in the art, and are intended to be within the scope of the present invention. The following is an example demonstrating one method of such a transformation. The material described herein is not intended to be limiting, but merely exemplary of one transformation technique. A VCG can be obtained in the following manner by the transformation of an ECG recorded simultaneously from a pair of electrodes. The ECG to VCG transformation calculations are presented here for all three lead pair combinations from a common three-lead relationship, but it should be noted that only one lead pair is required to generate the VCG. Also, for the following, at any time (t), the magnitude (voltage) of the recording for Lead I(t) is defined as I, the magnitude (voltage) of the recording for Lead II(t) is defined as II, and the magnitude (voltage) of the recording for Lead III(t) is defined as III.

For the Lead I and II combination, the first task is to define the angle (θ) and magnitude (E) of the VCG at time (t), from I and II. Since Lead I is at 0° and Lead II is at 60° (see FIG. 1), E is the vector addition of the values along Leads I and II. Assume E is at angle θ. Then:
I=E cos (θ) Equation 1
and
II=E cos (60−θ) Equation 2
Now, since cos (A−B)=cos (A) cos (B)+sin (A) sin (B), we have
II=(E/2)[cos (θ)+\sqrt{3} sin (θ)] Equation 3
Combining Equations 1 and 3, we get:
II/I=(½)[1+\sqrt{3} tan (θ)] Equation 4
And thus:

$$θ = {tan}^{- 1} ( \frac{2 II - I}{\sqrt{3} I} ) \qquad Equation 5$$

Next calculate the sin (θ) and the cos (θ). Since the hypotenuse of θ is \sqrt{[2II−I]²+[\sqrt{3}I]²}, or in simplified form:
hypotenuse(θ)=\sqrt{4II²−4I(II)+4I²} Equation 6
Then

$$\begin{matrix}cos ( θ ) = \frac{\sqrt{3} I}{2 \sqrt{{II}^{2} - I ( II ) + {I}^{2}}} \\ And & Equation 7 \\ sin ( θ ) = \frac{2 II - I}{2 \sqrt{{II}^{2} - I ( II ) + {I}^{2}}} \\ From which & Equation 8 \\ E = \frac{2 \sqrt{{II}^{2} - I ( II ) + {I}^{2}}}{\sqrt{3}} & Equation 9\end{matrix}$$

For the generation of the VCG, the following can be used:

1. The measurements for I(t) and II(t) for the two leads.

2. Equation 5 to determine angle θ.

3. Equation 9 to determine the magnitude, E.

4. The value I is the x-vertex.

5. The y-vertex is computed from Equation 10.
y=E cos (90−θ)=E sin (θ) Equation 10
Then, for each time (t) sample, an (x,y) vertex is generated, and hence the vectorcardiogram.

For the Lead I and III combination, the first task is to define the angle (θ) and magnitude (E) of the VCG at time (t), from I and III. Since Lead I is at 0° and Lead III is at 120° (see FIG. 1), E is the vector addition of the values along Leads I and III. Assume E is at angle θ. Then:
I=E cos (θ) Equation 11
And
III=E cos (120−θ) Equation 12
Now, since cos (A−B)=cos (A) cos (B)+sin (A) sin (B), we have
III=(E/2)[\sqrt{3} sin (θ)−cos (θ)] Equation 13
Combining Equations 11 and 13, we get:
III/I=(½)[\sqrt{3} tan (θ)−1] Equation 14
And thus:

$$θ = {tan}^{- 1} ( \frac{2 III + I}{\sqrt{3} I} ) \qquad Equation 15$$

Next calculate the sin (θ) and the cos (θ). Since the hypotenuse of θ is \sqrt{[2III+I]²+[\sqrt{3}I]²}, or in simplified form:

$$\begin{matrix}hypotenuse ( θ ) = \sqrt{4 {III}^{2} + 4 I ( III ) + 4 {I}^{2}} \\ Then & Equation 16 \\ cos ( θ ) = \frac{\sqrt{3} I}{2 \sqrt{{III}^{2} + I ( III ) + {I}^{2}}} \\ And & Equation 17 \\ sin ( θ ) = \frac{2 III - I}{2 \sqrt{{III}^{2} + I ( III ) + {I}^{2}}} \\ From which & Equation 18 \\ E = \frac{2 \sqrt{{III}^{2} + I ( III ) + {I}^{2}}}{\sqrt{3}} & Equation 19\end{matrix}$$

For the generation of the VCG, the following can be used:

1. The measurements for I(t) and III(t) for the two leads.

2. Equation 15 to determine angle θ.

3. Equation 19 to determine the magnitude, E.

4. The value I is the x-vertex.

5. The y-vertex is computed from Equation 20.
y=E cos (90−θ)=E sin (θ) Equation 20
Then, for each time (t) sample, an (x,y) vertex is generated, and hence the vectorcardiogram.

For the Lead II and III combination, the first task is to define the angle (θ) and magnitude (E) of the VCG, at time (t), from II and III. Since Lead II is at 60° and Lead III is at 120° (see FIG. 1), E is the vector addition of the values along Leads II and III. Assume E is at angle θ. Then:
II=E cos (60−θ) Equation 21
And
III=E cos (120−θ) Equation 22
Now, since cos (A−B)=cos (A)cos (B)+sin (A) sin (B), we have
II=(E/2)[cos (θ)+\sqrt{3} sin (θ)] Equation 23
III=(E/2)[\sqrt{3} sin (θ)−cos (θ)] Equation 24
Combining Equations 23 and 24, we get:

$$\begin{matrix}III / II = \frac{\sqrt{3} tan ( θ ) - 1}{\sqrt{3} tan ( θ ) + 1} \\ And thus \text{:} & Equation 25 \\ θ = {tan}^{- 1} [ \frac{II + III}{\sqrt{3} ( II - III )} ] & Equation 26\end{matrix}$$

Next calculate the sin (θ) and the cos (θ). Since the hypotenuse of θ is \sqrt{[II+III]²+[\sqrt{3}(II−III)]²}, or in simplified form:

$$\begin{matrix}hypotenuse ( θ ) = \sqrt{4 {II}^{2} - 4 II ( III ) + 4 {III}^{2}} \\ Then & Equation 27 \\ cos ( θ ) = \frac{\sqrt{3} ( II - III )}{2 \sqrt{{II}^{2} - II ( III ) + {III}^{2}}} \\ And & Equation 28 \\ sin ( θ ) = \frac{II + III}{2 \sqrt{{II}^{2} - II ( III ) + {III}^{2}}} \\ From which & Equation 29 \\ E = \frac{2 \sqrt{{II}^{2} - II ( III ) + {III}^{2}}}{\sqrt{3}} & Equation 30\end{matrix}$$

For the generation of the VCG, the following can be used:

1. The measurements for II(t) and III(t) for the two leads.

2. Equation 26 to determine angle θ.

3. Equation 30 to determine the magnitude, E.

4. The value x-vertex is computed from Equation 31.
x=E cos (θ) Equation 31

5. The y-vertex is computed from Equation 32.
y=E cos (90−θ)=E sin (θ) Equation 32
Then, for each time (t) sample, an (x,y) vertex is generated, and hence the vectorcardiogram.

In one aspect of the present invention, filtering the VCG includes reducing a VCG signal artifact. Various types of artifacts may be present in the VCG, including electrical noise, thermal noise, movement artifacts, breathing artifacts, and combinations thereof. The following is a description of a few types of noise artifacts that are often present. It should be noted, however, that any type of noise capable of being filtered from the signal is considered to be within the scope of the present invention.

One common type of signal artifact is power line noise. This type of noise is a result of the AC frequency of the power lines being picked up by the recording leads. The signal is about 60 Hz in the United States, and about 50 Hz in Europe. Any means of performing a time-to-frequency transformation can be used to find the line frequency component, including the discrete Fourier transform (DFT), which is well known to one skilled in the art. The 50/60 Hz component can be directly assessed by locating a 50 or 60 Hz peak in the frequency spectrum. In the attenuation of power line noise, it is useful to note that although the presence of a 50/60 Hz peak may originate from a biological signal, it will not be of a constant phase relationship when viewed in the VCG domain, while a 50/60 Hz peak from power line noise will be of a constant phase relationship. As such, identification of power line noise may be accomplished by examination of frequency and phase relations. As an aside, if performance is restricted, the 50/60 Hz artifacts can be processed with breathing/motion artifact filtering, as discussed herein.

Another common type of signal artifact is referred to as DC shift or DC drift. Because different electrode combinations, and thus different leads, will have different relative ground values, the mean voltage on the leads can differ. When the mean value is significantly different from zero and/or the mean value of a clean signal, the lead is said to have a DC drift. For simplicity, DC drift is the magnitude of the mean value of the voltage on any particular lead. In order to detect DC drift, the mean value of a signal is measured over a reasonable time period, e.g., 1000 msec. If the measured value is greater than a small percentage of the peak-to-peak signal range (highest voltage to lowest voltage over the interval), then DC drift is present. The severity of the DC drift can be estimated from the equation: |mean value of voltage|/(+supply voltage).

Yet another common type of signal artifact is referred to as saturation. Sensors and analogue-to-digital converters have a range of values to which they typically respond, which is often determined by the supply voltage, e.g. +/−1.5. Sampled signal values at either end of this range are considered “saturated” because their actual values are outside of the range of the signal recording equipment. These saturated signals appear to have portions that are “clipped” or “cropped” off at the upper and/or lower range. Saturation can be assessed by looking for signals that: 1) are within 10% of the +/−supply voltage (postamplification); 2) are consistent from one sample to the next; and 3) have a low variance. It should be noted that saturation will rarely be exactly the same as the supply voltage. In some cases, 5-10% variation in the actual +/−supply voltage will be commonplace.

One of the most significant sources of signal artifacts results from breathing, muscular movements, and other motion artifacts of the patient during cardiac signal recording. Breathing typically occurs between 10-20 times per minute, and thus has a spectral density (ESD) magnitude in the range of 0.17-0.33 Hz. Muscular (electromyogram, or EMG) and motion artifacts have a higher frequency content than breathing artifacts, and tend to spread throughout much of the measured frequency spectrum. A variety of techniques for assessing breathing/motion artifacts can be utilized in the present invention. The methods described hereafter are not intended to be limiting, and may also be utilized to reduce any type of periodic signal artifact. One method is the use of a simple variance. The variance is the sum of the squared error from the mean of the signal, divided by the number of samples. Signals may be used containing DC drift, although saturated signals may be eliminated. Since this method does not distinguish between one part of the time window and another, a signal with an episode of very high noise will have a similar variance to a signal with moderate noise throughout the time window.

Another method of assessing breathing/motion artifacts is examines the summed score of the sub-interval variance. In this method, the time window, e.g., 1000 msec, is divided into sub-intervals, e.g., 25 msec, and the variance assessed. Variance above the normal 50% peak-to-peak range of the biological signal is considered “high”, and scored as one point for every multiple it is of the 50% peak-to-peak range. In a normal ECG, only the QRS complex will cause a point to be recorded. Given average heart rates of approximately 1-3 beats per second, the summed score will typically be from 1-3. Breathing/motion artifacts will cause this score to climb above 10, thus indicating the presence of cyclic signal noise.

Yet another method of assessing breathing/motion artifacts examines the percent of sub-intervals with high variance. In this method, a time window is divided into sub-intervals as described above. In this case, the number of sub-intervals with a high variance is divided by the number of sub-intervals. This corrects for differences in the absolute value of the QRS complex, and provides normalization across leads of differing orientation.

Any means of filtering a signal artifact from a VCG should be considered within the scope of the present invention. As such, the filtering examples described herein are merely illustrative, and are not intended to be limiting. For example, the current VCG may be filtered by using a previous or other VCG as a template for the current VCG, whereby the current VCG is fit to the template by eliminating extreme outliers until a stable smooth curve is obtained. One method for accomplishing this is through recursive curve fitting (nonlinear regression).

The VCG can also be filtered by determining the variability in the rate of change in the VCG data, including both the magnitude and direction, especially during the PQ, ST, and TP intervals of the VCG, to determine the type of noise present. Noise information can be used to clean up the P, QRS, and/or T loops. In other words, the VCG can be filtered specifically for the type of noise identified.

Noise artifacts can also be eliminated by checking for large instantaneous changes in the VCG magnitude and/or angle. Since the VCG is normally smooth, even under a variety of cardiac anomalies such as flutter and fibrillation, large instantaneous changes can be discarded and the remaining points in the curve can be fitted with interpolation, e.g., cubic spline, etc., to generate a noise free curve approximation. In one aspect, spikes in the VCG which result in instantaneous deviations of more than about 10% of the mean of the major/minor axes of the loop can be discarded. Also, iterative methods can be used. The VCG can also be filtered by replacing each value of the VCG curve with the moving average of the value and its surrounding values.

In order to provide the filtered VCG information in a more familiar form for physicians and other medical professionals, the filtered VCG can be transformed into a filtered ECG. A filtered ECG can be generated for each of the lead pairs previously used to generate the VCG. For example, ECGs for Leads I, II, and III, can be generated by calculating the magnitude (voltage) of the ECG for that lead at any time (t), using the magnitude (E) and the angle (θ) from the VCG in each of Equations 33, 34, and 35, respectively.
I=E cos (θ) Equation 33
II=E cos (60−θ) Equation 34
III=E cos (120−θ) Equation 35

The methods of the present invention also provide steps of diagnosing a patient condition. Diagnosis can occur by examination of the cardiac signal at any point along the process from acquiring an ECG to generating a filtered ECG. As such, in one aspect, the patient condition can be diagnosed by examination of the filtered VCG. In another aspect, the patient condition can be diagnosed by examination of the filtered ECG.

Of course, it is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications may be made without departing from the principles and concepts set forth herein.

What is claimed is:

1. A method of obtaining a filtered electrocardiogram, comprising steps of:
obtaining a vectorcardiogram;
filtering the vectorcardiogram to form a filtered vectorcardiogram; and
transforming the filtered vectorcardiogram into a filtered electrocardiogram.

2. The method of claim 1, wherein the step of obtaining the vectorcardiogram includes steps of:
obtaining an electrocardiogram; and
transforming the electrocardiogram into the vectorcardiogram.

3. The method of claim 2, wherein the electrocardiogram is obtained as a raw electrocardiogram.

4. The method of claim 2, wherein the electrocardiogram is obtained as a processed electrocardiogram.

5. The method of claim 2, wherein the step of obtaining the electrocardiogram further comprises steps of:
electrically associating at least two leads with a subject; and
recording electrocardiogram signals from the subject via the at least two leads.

6. The method of claim 5, wherein the step of electrically associating includes electrically associating at least three leads.

7. The method of claim 5, wherein the step of electrically associating includes electrically associating at least four leads.

8. The method of claim 6, further comprising a step of selecting a pair of leads that provides an acceptable level of signal quality for transforming the electrocardiogram into the vectorcardiogram.

9. The method of claim 8, wherein the pair of leads provide a higher level of signal quality than other pairs of leads of the at least three leads.

10. The method of claim 5, wherein the step of transforming the electrocardiogram into the vectorcardiogram occurs during an overlapping period of time with respect to the step of recording of the electrocardiogram signal.

11. The method of claim 2, wherein the step of obtaining the electrocardiogram includes obtaining the electrocardiogram from a storage location.

12. The method of claim 1, wherein the step of filtering the vectorcardiogram includes reducing a vectorcardiogram signal artifact.

13. The method of claim 12, wherein the signal artifact is a member selected from the group consisting of electrical noise, thermal noise, movement artifacts, breathing artifacts, and combinations thereof.

14. The method of claim 1, further comprising a step of diagnosing a patient condition.

15. The method of claim 14, wherein the step of diagnosing the patient condition includes an examination of the filtered vectorcardiogram.

16. The method of claim 14, wherein the step of diagnosing the patient condition includes an examination of the filtered electrocardiogram.
